(19) United States
(12) Patent Application Publication
(10) Pub. No.: US 2013/0004441 A1
(43) Pub. Date: Jan. 3, 2013

(54) NON-AQUEOUS STRUCTURED COMPOSITIONS

(75) Inventors: Hy Si Bui, Piscataway, NJ (US); Mohamed Kanji, Edison, NJ (US); Liana Esposito, Franklin Park, NJ (US); Kerrie Holguin, Honolulu, HI (US); Chunhua Li, Scotch Plains, NJ (US)

(73) Assignee: LOREAL, Paris (FR)

(21) Appl. No.: 13/583,677

(22) PCT Filed: Mar. 10, 2011
(86) PCT No.: PCT/US11/27866

§ 371 Date: Sep. 10, 2012

Related U.S. Application Data
(60) Provisional application No. 61/312,560, filed on Mar. 10, 2010.
(60) Provisional application No. 61/312,546, filed on Mar. 10, 2010.

(51) Int. Cl. A61K 8/72; A61K 47/30; A61Q 1/02; A61Q 1/10; C09D 11/00; A61Q 17/04; A61Q 15/00

(52) U.S. Cl. 424/59; 424/65; 514/772.3; 424/70.7; 106/31.13; 424/63; 512/4

(57) ABSTRACT

Disclosed are non-aqueous compositions capable of forming a gel structure, comprising (a) a low molecular mass N-acyl glutamic acid diamide having a straight-chain alkyl group; (b) a low molecular mass N-acyl glutamic acid diamide having a branched-chain alkyl group; (c) at least one gel-promoting solvent which includes a substituted hydrocarbyl functional siloxane and/or a second, different gel-promoting solvent; (d) at least one active ingredient, and (e) optionally, at least one polyorganosiloxane-containing polymer; wherein the composition has a hardness value ranging from about 30 to about 300 gf, a melting point of about 50° C. or higher. Methods of making and using the compositions are also disclosed.

### CROSS-REFERENCE TO RELATED APPLICATIONS

This application claims the benefit of the filing date of U.S. Provisional Patent Application Nos. 61/312,560, and 61/312,546, both filed Mar. 10, 2010, the disclosures of which are hereby incorporated herein by reference.

### FIELD OF THE INVENTION

The present invention relates, in general, to non-aqueous, structured, gel compositions, and more particularly, to non-aqueous, structured, gel compositions which possess good storage stability, particularly with respect to variations in temperature, which may be transparent or colored, have good payoff and do not require the use of wax as a structuring agent.

### BACKGROUND OF THE INVENTION

Conventional structured compositions typically employ various types of waxes as structuring agents in order to form user-friendly compositions having good pay-off (a term used to describe both the amount of composition applied onto a target substrate, as well as, the way the composition distributes onto the substrate), and stability properties, particularly with respect to temperature stability. The problem with wax-based stick compositions is that they possess an undesirable waxy feel and, if used to impart color, fail to facilitate significant color deposit onto a targeted keratinous substrate.

Attempts have been made in the past to formulate structured gel-form compositions in the absence of wax. For example, various types of polyamides have been commercialized as gellators/structuring agents in order to form solid compositions. Similarly, various glutamides, as well as various types of polyurethanes have also been commercialized in order to form solid, preferably transparent, compositions. Such attempts, however, while successful at making solid compositions, yielded numerous technical problems.

One of the technical problems associated with the above-referenced, commercial wax-free compositions involves their stability when exposed to elevated temperatures. It is imperative, from a practical point of view, that such compositions be able to withstand fluctuations in temperature during conventional storage conditions without their becoming too soft, thereby negatively impacting their use profile. In order to avoid such stability issues, the composition must possess a certain melting point profile.

Another technical problem relates to the way in which the composition is deposited onto a target substrate, also referred to “pay-off”. Poor pay-off, defined as too much deposit, too little deposit, or lack of uniformity of deposit, is primarily associated with the hardness/elasticity of the structured composition. Thus, in order to avoid such deposit issues, particularly with respect to color deposit, it is necessary that the composition possess certain hardness/elasticity properties.

It is therefore an object of the present invention to provide non-aqueous, structured, compositions that may be transparent or contain a colorant (and are capable of enhanced color deposit) and do not suffer from the aforementioned technical problems.

### BRIEF SUMMARY OF THE INVENTION

A first aspect of the present invention is directed to a non-aqueous composition that is capable of forming a gel structure, e.g., a soft gel or a hard or molded gel (such as a gel stick), that may be transparent or contain a colorant, and which includes (a) a low molecular mass N-acyl glutamic acid diamide having a straight-chain alkyl group; (b) a low molecular mass N-acyl glutamic acid diamide having a branched-chain alkyl group; (c) at least one gel-promoting solvent which includes a substituted hydrocarbyl functional siloxane and/or a second, different gel-promoting solvent; (d) at least one active ingredient, and (e) optionally, at least one polyorganosiloxane-containing polymer; wherein the composition has a hardness value ranging from about 30 to about 300 gf, a melting point of about 50° C. or higher, does not require use of wax as a structuring agent (e.g., is wax-free and exhibits enhanced pay-off).

In some embodiments, the non-aqueous compositions include (a) a low molecular mass N-acyl glutamic acid diamide having a straight-chain alkyl group; (b) a low molecular mass N-acyl glutamic acid diamide having a branched-chain alkyl group; (c) at least one solvent including at least one substituted hydrocarbyl functional siloxane; (d) optionally at least one additional gel-promoting solvent; (e) at least one polyorganosiloxane-containing polymer and (f) at least one active ingredient, wherein the composition has a hardness value ranging from about 30 to about 300 gf, a melting point of about 50° C. or higher, and does not require use of wax as a structuring agent.

In other embodiments, the non-aqueous compositions include a) a low molecular mass N-acyl glutamic acid diamide having a straight-chain alkyl group; b) a low molecular mass N-acyl glutamic acid diamide having a branched-chain alkyl group; c) at least one solvent; d) at least one polyorganosiloxane-containing polymer; e) at least one substituted hydrocarbyl functional siloxane; (f) optionally, at least one auxiliary solvent other than (c); and (g) at least one colorant, wherein the composition has a hardness value ranging from about 30 to about 300 gf, a melting point of about 50° C. or higher, does not require use of wax as a structuring agent (e.g., is free of wax), and is capable of enhanced color deposit.

A related aspect of the present invention is directed to a method of making the compositions, which entails the steps of mixing together the following ingredients: (a) a low molecular mass N-acyl glutamic acid diamide having a straight-chain alkyl group; (b) a low molecular mass N-acyl glutamic acid diamide having a branched-chain alkyl group; (c) at least one solvent including at least one substituted hydrocarbyl functional siloxane; (d) optionally at least one additional gel-promoting solvent; (e) at least one polyorganosiloxane-containing polymer and (f) at least one active ingredient, wherein the mixing is conducted at an elevated temperature generally ranging from about 90° C. to about 125° C., thus forming a heated composition and cooling the heated composition to form a non-aqueous, structured composition having a hardness value ranging from about 30 to about 300 gf, a melting point of about 50° C. or higher, and which does not require use of wax as a structuring agent (e.g., is wax-free). Preparing the compositions at these temperatures minimizes both the cost, and degree of manufacturing difficulty.

In other embodiments the process makes a non-aqueous colored (non-transparent) composition, and includes a) providing a first composition, comprising: i) a low molecular mass N-acyl glutamic acid diamide having a straight-chain alkyl group; ii) a low molecular mass N-acyl glutamic acid diamide having a branched-chain alkyl group; iii) at least one gel-promoting solvent; b) providing a second composition, comprising: iv) at least one polyorganosiloxane-containing polymer; v) at least one substituted hydrocarbyl functional siloxane; vi) optionally, at least one auxiliary solvent other than (c); and (vii) at least one colorant; c) mixing (a) and (b) at a temperature of generally ranging from about 90° C. to about 125° C., to form a heated composition; and d) cooling the heated composition to form the non-aqueous, structured, gel-form composition, wherein the composition has a hardness value ranging from about 30 to about 300 gf, a melting point of about 50° C. or higher, does not require use of wax as a structuring agent (e.g., is free of wax) and is capable of enhanced color deposit.

### BRIEF DESCRIPTION OF THE DRAWING

FIG. 1 is a color photograph of a product formed using a composition according to an embodiment of the present invention.

FIG. 2 is a color photograph of a composition according to an embodiment of the present invention.

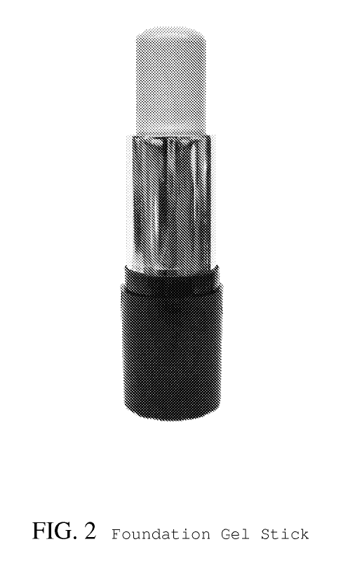

### DETAILED DESCRIPTION OF THE INVENTION

Other than in the operating examples, or where otherwise indicated, all numbers expressing quantities of ingredients and/or reaction conditions are to be understood as being modified in all instances by the term “about”, which as used herein refers to ±10-±15% of the referenced value.

As used herein, “structured” means gelled and/or rigidified.

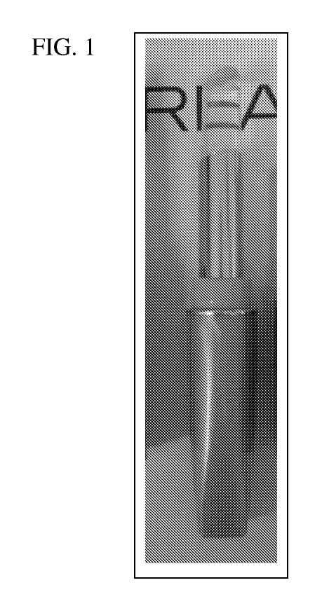

The term “transparent” is generally defined as having the property of transmitting rays of light through its substance so that bodies situated beyond or behind can be distinctly seen, as seen in FIG. 1.

It has been surprisingly discovered that the inventive compositions and processes yield a non-aqueous, structured, gel-form composition having excellent pay-off, do not require use of a wax as a structuring agent (e.g., they are wax-free), exhibit good storage stability, and which in some embodiments are transparent in appearance, and which in other embodiments, contain a colorant and have enhanced color deposit . . . .

### Gelators

The low molecular mass (wherein the term “low molecular mass” as used herein refers to a molecular mass from greater than zero up to about 2,000 daltons) gelators for use in the present invention are generally chosen from at least one N-acyl glutamic acid diamide having a straight-chain alkyl group, such as dibutyl lauroyl glutamide, and at least one N-acyl glutamic acid diamide having a branched-chain alkyl group, such as dibutyl ethylhexanoyl glut amide.

It has also surprisingly been discovered by the inventor that the combination of at least one N-acyl glutamic acid diamide having a straight-chain alkyl group, such as dibutyl lauroyl glutamide, with at least one N-acyl glutamic acid diamide having a branched-chain alkyl group, such as dibutyl ethylhexanoyl glutamide, facilitates the formation of a gel carrier composition having optimal physical properties.

The dibutyl lauroyl glutamide is present in an amount generally ranging from about 0.1 to about 10% by weight, such as from about 0.5 to about 5% by weight, and from about 1.0 to about 3% by weight, all weights being based on the total weight of the composition. For purposes of making the colored compositions of the present invention, the dibutyl lauroyl glutamide is employed in an amount generally ranging from about 0.1 to about 50% by weight, such as from about 0.2 to about 40% by weight, and from about 0.3 to about 30% by weight, all weights being based on the total weight of the first composition.

Similarly, the dibutyl ethylhexanoyl glutamide is employed in an amount of from about 0.1 to about 10% by weight, such as from about 0.5 to about 5% by weight, and from about 1 to about 3% by weight, all weights being based on the total weight of the composition. For purposes of making the colored compositions of the present invention, the dibutyl ethylhexanoyl glutamide is employed in an amount generally ranging from about 0.1 to about 50% by weight, such as from about 0.2 to about 40% by weight, and from about 0.3 to about 30% by weight, all weights being based on the total weight of the first composition.

In order to make the embodiments of the inventive compositions that are clear or transparent in appearance (in which case they contain no pigment or less than about 0.5% pigment), the composition (in addition to being free of colorant) should employ the low molecular mass organogellators in a total amount of less than about 7% by weight, based on the weight of the composition.

The dibutyl lauroyl glutamide is commercially available as GP-1 and the dibutyl ethylhexanoyl glutamide is commercially available as EB-21, both available from Ajinomoto of Fort Lee, N.J.

In a preferred embodiment, the N-acyl glutamic acid diamide having a straight chain alkyl group and the N-acyl glutamic acid diamide having a branched chain alkyl group are employed in a ratio by weight of from about 1:1 to about 3:1, and preferably about 1.5:1.

### Polyorganosiloxane-Containing Polymer

The compositions may contain a polyorganosiloxane-containing polymer, which as disclosed herein is a polymer (homopolymer or copolymer) having at least one moiety which contains: at least one polyorganosiloxane group consisting of 1 to about 1000 organosiloxane units in the chain of the moiety or in the form of a graft, and at least two groups capable of establishing hydrogen interactions. The polyorganosiloxane is preferably present in the colored compositions due to its excellent pay-off properties, but it may also be present in the transparent compositions because it provides additional structuring properties.

The polyorganosiloxane-containing polymers may comprise at least one moiety corresponding to formula (I):

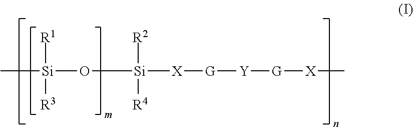

in which:
1) R¹, R², R³ and R⁴, which may be identical or different, represent a group chosen from:

(a) linear, branched or cyclic, saturated or unsaturated, C₁ to C₄₀ hydrocarbon-based groups, possibly containing in their chain one or more oxygen, sulphur and/or nitrogen atoms, and possibly being partially or totally substituted with fluorine atoms,

(b) C₆ to C₁₀ aryl groups, optionally substituted with one or more C₁ to C₄ alkyl groups,

(c) polyorganosiloxane chains possibly containing one or more oxygen, sulphur and/or nitrogen atoms;

2) X, which may be identical or different, represents a linear or branched C₁ to C₃₀ alkylenediyl group, possibly containing in its chain one or more oxygen and/or nitrogen atoms;
3) Y is a saturated or unsaturated, C₁ to C₅₀ linear or branched divalent alkylene, arylene, cycloalkylene, alkylarylene or arylalkylene group, optionally comprising one or more oxygen, sulphur and/or nitrogen atoms, and/or optionally substituted with one of the following atoms or groups of atoms: fluorine, hydroxyl, C₃ to C₈ cycloalkyl, C₁ to C₄₀ alkyl, C₅ to C₁₀ aryl, phenyl optionally substituted with one to three C₁ to C₃ alkyl, C₁ to C₃ hydroxyalkyl, and C₁ to C₆ aminoalkyl groups;
4) G, which may be identical or different, represents a group chosen from ester, amide, sulphonamide, carbamate, thiocarbamate, urea, thiourea groups, and combinations thereof;
5) m is an integer ranging from 1 to 1,000, preferably from 1 to 700 and more preferably from 6 to 200; and
6) n is an integer ranging from 2 to 500 and preferably from 2 to 200.
The polyorganosiloxane-containing polymers may also comprise at least one moiety corresponding to formula (II):

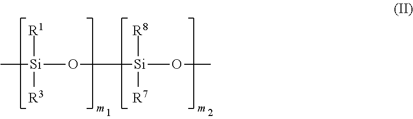

in which

R¹ and R³, which may be identical or different, are as defined above for formula (I),

R⁷ represents a group as defined above for R¹ and R³, or represents a group of formula —X-G-R⁹ in which X and G are as defined above for formula (I) and R⁹ represents a hydrogen atom or a linear, branched or cyclic, saturated or unsaturated, C₁ to C₅₀ hydrocarbon-based group optionally comprising in its chain one or more atoms chosen from O, S and N, optionally substituted with one or more fluorine atoms and/or one or more hydroxyl groups, or a phenyl group optionally substituted with one or more C₁ to C₄ alkyl groups,

R⁸ represents a group of formula —X-G-R⁹ in which X, G and R⁹ are as defined above,

m₁ is an integer ranging from 1 to 998, and

m₂ is an integer ranging from 2 to 500.

According to another embodiment, it is also possible to use a copolymer comprising several different moieties of formula (I), and/or several different moieties of formula (II), that is to say a polymer in which at least one of the groups R¹, R², R³, R⁴, X, G, Y, m and n is different in one of the moieties.

It is also possible to use a copolymer comprising at least one moiety of formula (I) and at least one moiety of formula (II), the moieties of formula (I) and the moieties of formula (II) possibly being identical to, or different from, each other. These copolymers may be block copolymers or grafted copolymers.

Additional polyorganosiloxane-containing polymers which may be used in the composition of the invention include those described in documents U.S. Pat. Nos. 5,874,069; 5,919,441; 6,051,216; and 5,981,680, the entire contents of which are hereby incorporated by reference.

A preferred polyorganosiloxane-containing polymer for use in the present invention will have at least one moiety chosen from formula (III):

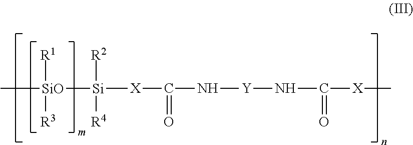

and formula (IV)

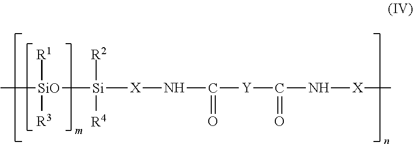

in which:
(a) R¹, R², R³ and R⁴ are the same or different and may be selected from the group consisting of methyl, ethyl, propyl, isopropyl, a siloxane chain, and phenyl;
(b) X is a linear or branched chain alkylene having 1-30 carbons;
(c) Y is selected from the group consisting of linear or branched chain alkylenes having 1-40 carbons;
(d) m is a number between 1 and 700;
(e) n is a number between 1 and 500.

Particularly preferred polyorganosiloxane-containing polymers useful herein are commercially available from Dow Corning as DC 8178 and DC 8179, which are known under the INCI denomination of Nylon-611/Dimethicone Copolymer.

The amount of the polyorganosiloxane-containing polymer used to make the colored compositions according to the invention generally ranges from about 1 to 40% by weight, preferably from 2 to 30% by weight, and more preferably from 3 to 20% by weight, based on the total weight of the second composition that is prepared for the purpose of making the colored compositions. The polyorganosiloxane-containing polymer is present in the resultant colored compositions in an amount generally ranging from about 1 to about 10% by weight, such as from about 2 to about 8% by weight, and from about 3 to about 6% by weight, all weights being based on the total weight of the resultant colored composition.

### Gel-Promoting Solvents

Particularly preferred solvents for use in the compositions of the present invention are substituted hydrocarbyl functional siloxanes. They are believed to promote hydrogen bond formation between molecules of the glutamides. They may also function as solvents for the polyorganosiloxane-containing polymer when it is present.

The hydrocarbyl functional organopolysiloxane of the present invention comprises a siloxy unit of the formula R¹RₐSiO_{(3-a)/2} wherein R is any monovalent hydrocarbon group, but typically is an alkyl, cycloalkyl, alkenyl, aralkyl, or an aryl group containing 1-20 carbon atoms, R₁ is a hydrocarbyl group having the formula —R²OCH₂CH₂OH, R² is a divalent hydrocarbon group containing 2 to 6 carbon atoms, a is zero to 2.

In some embodiments, the siloxanes of the present invention have a formula selected from the group consisting of:

### R₃SiO(RR¹SiO)_{y}SiR₃,

### R₃SiO(R₂SiO)ₓ(RR¹SiO)_{y}SiR₃,

### R¹R₂SiO(R₂SiO)ₓSiR₂R¹,

### R¹R₂SiO(RR¹SiO)_{z}SiR₂R¹,

### R¹R₂SiO(R₂SiO)ₓ(RR¹SiO)_{z}SiR₂R¹,

### R¹R₂SiO(R₂SiO)ₓSiR₃,

### R¹R₂SiO(RR¹SiO)_{z}SiR₃,

R¹R₂SiO(R₂SiO)ₓ(RR¹SiO)_{z}SiR₃, and cyclic siloxanes of the formula -(Me₂SiO)ₙ(MeR¹SiO)ₙ—

In these formulas, R is an alkyl, cycloalkyl, alkenyl, aralkyl, or an aryl group containing 1-20 carbon atoms; R¹ is the hydrocarbyl group as defined above, e.g., a group having one of the formulas (i)-(iv):

—(CH₂)ₐ(OCH₂CH₂)OR² (i)

—(CH₂)ₐ(OCH₂CH₂CH₂)OR² (ii)

—(CH₂)ₐ[OCH₂CH(CH₂CH₃)]OR² (iii)

—(R)_{b}OR² (iv)

wherein a is 3-11; b is 1-50; R² is selected from the group consisting of hydrogen, an alkyl group, an aryl group, an arylalkyl group and an acyl group; x is 1-500, y is 1-40, z is 1-40, m is 1-6, n is 1-6, and the sum of m+n is 3-12. See, e.g., U.S. Patent Application Publication 2004/0223936 A1.

For the sake of simplicity in nomenclature in the Examples, these substituted hydrocarbyl siloxanes are referred to as the CARBINOL FLUID, bis-hydroxyethoxypropyl dimethicone, which is a hydrocarbyl functional organopolysiloxane having the formula, R¹Me₂SiO(Me₂SiO)ₓSiMe₂R¹ where R¹ is —(CH₂)₃OCH₂CH₂OH, and x is such to provide the product with a viscosity of about 50 cS (mm₂/s) at 23° C.

The term substituted hydrocarbyl is therefore intended to mean any such hydrocarbyl group wherein at least one hydrogen atom has been substituted with an atom other than hydrogen, or with a group of atoms containing at least one atom other than hydrogen. For example, the hydrogen atom can be substituted with a halogen atom such as a chlorine or fluorine atom. The hydrogen atom alternatively can be substituted with an oxygen atom, or with a group containing an oxygen atom to form a hydroxy group, an ether, an ester, an anhydride, an aldehyde, a ketone, or a carboxylic acid. The hydrogen atom also can be replaced with a group containing a nitrogen atom to form an amide or a nitro group. In addition, the hydrogen atom can be substituted with a group containing a sulfur atom to form —SO₃H.

The substituted hydrocarbyl functional siloxanes of the present invention can be made by standard processes such as the hydrosilylation of organohydrogensiloxanes and olefinically substituted polyoxyalkylenes. The hydrosilylation reaction is typically performed in a low molecular weight volatile hydrocarbon solvent such as benzene, toluene, xylene, or isopropanol to aid in handling the reactants, to moderate an exothermic reaction or to promote the solubility of the reactants.

In some embodiments, the substituted hydrocarbyl functional siloxane is typically present in the transparent compositions in an amount ranging from 10 to 98% by weight, preferably from 15 to 85% by weight, more preferably from 20 to 75% by weight, based on the total weight of the resultant composition. In other embodiments, the substituted hydrocarbyl functional siloxane is present in an amount generally ranging from about 5 to about 50% by weight, such as from about 10 to about 40% by weight, and from about 15 to about 30% by weight, all weights being based on the total weight of the resultant composition. The substituted hydrocarbyl functional siloxane may be present in an amount ranging from 5 to 80% by weight, preferably from 10 to 60% by weight, more preferably from 15 to 50% by weight, based on the weight of the second composition.

Other gel-promoting solvents useful in the present invention, that may be the sole solvent or in preferred embodiments is combined with the substituted hydrocarbyl siloxane may include both polar and non-polar solvents. Suitable solvents include, for example, alcohols, monoalcohols, dialcohols, acids, esters, and the like. Polar solvents are believed to promote gel formation by inhibiting intercalation (intramolecular bonding) in the glutamide molecules.

Preferred polar solvents include, but are not limited to, C2-C5 glycols, such as propylene glycol, butylene glycol and pentylene glycol. Other preferred solvents include, for example, octododecanol, isostearyl alcohol, and the like. The preferred solvents listed herein may be used individually or in combination with other suitable solvents. These solvents may be present as additional hydrogen bond-forming solvents in the transparent compositions.

It is preferred that the solvents be capable of dissolving the organogellators at a temperature of from about 90° C. to about 125° C.

These gel-promoting solvents are typically present (in preferred embodiments as a second solvent) in an amount of from about 1 to about 20% by weight, such as from about 2 to about 10% by weight, and from about 3 to about 5% by weight, all weights being based on the total weight of the resultant composition. When present as the sole solvent, they may be present in an amount ranging from 10 to 98% by weight, preferably from 15 to 85% by weight, more preferably from 20 to 75% by weight, based on the total weight of the resultant composition. These gel-promoting solvents are typically present in an amount of from about 40-70% by weight, such as from about 40-60% by weight, and from about 40-50% by weight used to form the first composition.

### Auxiliary Solvents

An auxiliary solvent may be present in the compositions of the present invention. Examples thereof include volatile and/or non-volatile oils. Such oils can be any cosmetically acceptable oil including but not limited to silicone oils and/or hydrocarbon oils.

According to preferred embodiments, the auxiliary solvent comprises one or more volatile silicone oils. Examples of such volatile silicone oils include linear or cyclic silicone oils having a viscosity at room temperature less than or equal to 6 cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C. Preferably, the volatile silicone oils have a flash point of at least 40° C.

Non-limiting examples of volatile silicone oils are listed in Table 1 below.

TABLE 1

| | Flash | |
|---|---|---|
| | Point | Viscosity |
| Compound | (° C.) | (cSt) |
| Octyltrimethicone | 93 | 1.2 |
| Hexyltrimethicone | 79 | 1.2 |
| Decamethylcyclopentasiloxane | 72 | 4.2 |
| (cyclopentasiloxane or D5) | | |
| Octamethylcyclotetrasiloxane | 55 | 2.5 |
| (cyclotetradimethylsiloxane or | | |
| D4) | | |
| Dodecamethylcyclohexasiloxane | 93 | 7 |
| (D6) | | |
| Decamethyltetrasiloxane(L4) | 63 | 1.7 |
| KF-96 A from Shin Etsu | 94 | 6 |
| PDMS (polydimethylsiloxane) DC | 56 | 1.5 |
| 200 (1.5 cSt) from Dow Corning | | |
| PDMS DC 200 (2 cSt) from Dow | 87 | 2 |
| Corning | | |

Volatile silicone oils that are linear may be employed in the present invention. Suitable volatile linear silicone oils include those described in U.S. Pat. No. 6,338,839 and WO03/042221, the contents of which are incorporated herein by reference. In one embodiment the volatile linear silicone oil is decamethyltetrasiloxane. In another embodiment, the decamethyltetrasiloxane is further combined with another solvent that is more volatile than decamethyltetrasiloxane.

According to other preferred embodiments, the auxiliary solvent includes one or more non-silicone volatile oils such as volatile hydrocarbon oils, volatile esters and volatile ethers. Examples of such volatile non-silicone oils include, but are not limited to, volatile hydrocarbon oils having from 8 to 16 carbon atoms and their mixtures and in particular branched C₈ to C₁₆ alkanes such as C₈ to C₁₆ isoalkanes (also known as isoparaffins), isodecane, and for example, the oils sold under the trade names of Isopar or Permethyl. Preferably, the volatile non-silicone oils have a flash point of at least 40° C.

Other non-limiting examples of volatile non-silicone oils are given in Table 2 below.

TABLE 2

| | Flash |
|---|---|
| | Point |
| Compound | (° C.) |
| Isododecane | 43 |
| Propylene glycol n-butyl ether | 60 |
| Ethyl 3-ethoxypropionate | 58 |
| Propylene glycol methylether | 46 |
| acetate | |
| Isopar L (isoparaffin C₁₁-C₁₃) | 62 |
| Isopar H (isoparaffin C₁₁-C₁₂) | 56 |

Examples of non-volatile oils that may be used as auxiliary solvents in the present invention include, but are not limited to, polar oils such as:

a.—hydrocarbon-based plant oils with a high triglyceride content consisting of fatty acid esters of glycerol, the fatty acids of which may have varied chain lengths, these chains possibly being linear or branched, and saturated or unsaturated; these oils are especially wheat germ oil, corn oil, sunflower oil, karite butter, castor oil, sweet almond oil, macadamia oil, apricot oil, soybean oil, rapeseed oil, cottonseed oil, alfalfa oil, poppy oil, pumpkin oil, sesame seed oil, marrow oil, avocado oil, hazelnut oil, grape seed oil, blackcurrant seed oil, evening primrose oil, millet oil, barley oil, quinoa oil, olive oil, rye oil, safflower oil, candlenut oil, passion flower oil or musk rose oil; or caprylic/capric acid triglycerides, for instance those sold by the company Stearineries Dubois or those sold under the names Miglyol 810, 812 and 818 by the company Dynamit Nobel;
b.—synthetic oils or esters of formula R₅COOR₆ in which R₅ represents a linear or branched higher fatty acid residue containing from 1 to 40 carbon atoms, including from 7 to 19 carbon atoms, and R₆ represents a branched hydrocarbon-based chain containing from 1 to 40 carbon atoms, including from 3 to 20 carbon atoms, such as, for example, Purcellin oil (cetostearyl octanoate), isononyl isononanoate, C₁₂ to C₁₅ alkyl benzoate, isopropyl myristate, 2-ethylhexyl palmitate, and octanoates, decanoates or ricinoleates of alcohols or of polyalcohols; hydroxylated esters, for instance isostearyl lactate or diisostearyl malate; and pentaerythritol esters;
c.—synthetic ethers containing from 10 to 40 carbon atoms;
d.—C₈ to C₂₆ fatty alcohols, for instance oleyl alcohol; and
e.—mixtures thereof.

Examples of non-volatile, non-polar oils that may be used as auxiliary solvents in the present invention include, but are not limited to, oils such as branched and unbranched hydrocarbons and hydrocarbon waxes including polyolefins, in particular Vaseline (petrolatum), paraffin oil, squalane, squalene, hydrogenated polyisobutene, hydrogenated polydecene, polybutene, mineral oil, pentahydrosqualene, and mixtures thereof.

The auxiliary solvent may be employed in an amount of from about 5 to about 80% by weight, such as from about 10 to about 70% by weight, and all subranges therebetween, all weights based on the total weight of the composition.

### Active Ingredients

The compositions contain at least one active ingredient. The compositions may contain a colorant, e.g., pigments, inks and lakes. Representative examples of other suitable active ingredients include, for example, dermatological ingredients such as sunscreen agents, anti-acne agents, anti-aging compounds; insect repelling agents; transdermal pharmaceutical compounds; deodorant and antiperspirant agents; perfumes; dye compounds; moisturizing agents; etc.

The type and amount of active ingredient to be employed will depend on the ultimate use of the composition, and can thus be determined by those of ordinary skill in the art. The active ingredient is present in amounts generally ranging from about 0.01 to 20 wt % and in some embodiments from about 0.1 to about 10% by weight, based on the total weight of the composition. As stated above, the compositions of the invention that are transparent may contain no colorant or colorant in an amount less than about 0.5% by weight. Compositions that contain colorant and which are colored in appearance will generally contain more than about 0.5% colorant.

The compositions of the present invention should be stable under conventional storage conditions. In order to achieve storage stability, the composition must have a melting point of about 50° C. or higher, such as 70° C. or higher, or even 80° C., 90° C. or higher.

The compositions should also have good “pay-off”, i.e., the ability to be elegantly and uniformly deposited onto a targeted substrate. This property is dependent on the hardness of the composition. The hardness of the composition may, for example, be expressed in gramforce (gf). The compositions of the present invention may, for example, have a hardness ranging from about 30 gf to about 300 gf, such as from about 50 gf to about 120 gf, and further such as from about 60 gf to about 100 gf.

Hardness is measured in one of two ways. A first test for hardness entails penetrating a probe into the composition and in particular using a texture analyzer (for example TA-XT21 from Rheo) equipped with an ebonite cylinder of height 25 mm and diameter 8 mm. The hardness measurement is carried out at 20° C. at the center of 5 samples of the composition. The cylinder is introduced into each sample of composition at a pre-speed of 2 mm/s and then at a speed of 0.5 mm/s and finally at a post-speed of 2 mm/s, the total displacement being 1 mm. The recorded hardness value is that of the maximum peak observed. The measurement error is ±50 gf.

The second test for hardness is known as the “cheese wire” method, which involves cutting an 8.1 mm or preferably 12.7 mm in diameter stick composition and measuring its hardness at 20° C. using a DFGHS 2 tensile testing machine from Indelco-Chatillon Co. at a speed of 100 mm/minute. The hardness value obtained from this method is expressed in grams as the shear force required to cut a stick under the above conditions. According to this method, the hardness of compositions according to the present invention which may be in stick form may, for example, range from 30 gf to 300 gf, such as from 30 gf to 250 gf, for a sample of 8.1 mm in diameter stick, and further such as from 30 gf to 200 gf, and also further such as from 30 gf to 120 gf for a sample of 12.7 mm in diameter stick.

The hardness of the composition of the present invention may be such that the compositions are self-supporting and can easily disintegrate to form a satisfactory deposit on a targeted substrate. In addition, this hardness may impart good impact strength to the inventive compositions, which may be molded, cast, or extruded, for example, in stick or dish form.

One use for the transparent, non-aqueous compositions of the present invention is as a personal care product for depositing at least one active ingredient onto a targeted keratinous substrate. Thus, the composition may be used as a lip gloss, transparent mascara, a hair styling composition, deodorant and the like.

FIG. 1 is a color photograph of a product formed using a composition according to an embodiment of the present invention. This photograph shows a product that is transparent. It can be seen that the letters placed behind product are visible through the transparent product.

The purpose of the non-aqueous colored compositions of the present invention is to impart color onto a targeted keratinous substrate. Thus, the colored compositions may be used as a foundation, mascara, and the like.

### Processes

The process for making the non-aqueous compositions involves: providing (a) a low molecular mass N-acyl glutamic acid diamide having a straight-chain alkyl group; (b) a low molecular mass N-acyl glutamic acid diamide having a branched-chain alkyl group; (c) at least one solvent including at least one substituted hydrocarbyl functional siloxane; (d) optionally at least one additional gel-promoting solvent; (e) at least one polyorganosiloxane-containing polymer and (f) at least one active ingredient; mixing (a)-(f), at an elevated temperature generally ranging from about 90° C. to about 125° C., to form a heated composition; and cooling the heated composition to form the non-aqueous, structured transparent composition.

In embodiments that entail forming first and second compositions, their respective ingredients are combined at an elevated temperature generally ranging from about 90 to about 125° C., followed by combining the first and second compositions at about the same temperature, e.g., from about 90 to about 125° C. The first and second compositions can be formed either concurrently or consecutively.

The present invention is now described in terms of the examples which follow, all of which are intended for illustrative purposes only, and are not meant to unduly limit the scope of the invention in any way.

### EXAMPLES

### Transparent Compositions and Processes

### Examples 1-3

| INCI Name | Ex 1 | EX 2 | EX 3 |
|---|---|---|---|
| PROPYLENE GLYCOL | 97.5 | 0 | 0 |
| BUTYLENE GLYCOL | 0 | 97.5 | 0 |
| PENTYLENE GLYCOL | 0 | 0 | 97.5 |
| GP-1 (dibutyl lauroyl | 1.5 | 1.5 | 1.5 |
| glutamide) | | | |
| EB-21 (dibutyl | 1 | 1 | 1 |
| ethylhexanoyl | | | |
| glutamide) | | | |
| TOTAL | 100 | 100 | 100 |
| Physical Property | SOFT | SOFT | LIQUID |
| | GEL | GEL | |
| STICK HARDNESS (gf) | Not | Not | No stick |
| | measured | measured | formation |

1. Heated glycol to around 100° C., then added gelators GP-1 and EB-21 gradually during mixing until they were dissolved to form a clear solution.
2. Poured the hot solution into the mold. Once the solution set in the mold, placed the mold in the freezer for about 30 minutes.

### Examples 4-10

### Synergy of Glycol and Carbinol Fluid Mixtures in the Hardness of the Gel Sticks

| INCI Name | Ex 4 | Ex 5 | EX 6 | EX 7 |
|---|---|---|---|---|
| PROPYLENE GLYCOL | 0 | 5 | 0 | 0 |
| BUTYLENE GLYCOL | 0 | 0 | 5 | 0 |
| PENTYLENE GLYCOL | 0 | 0 | 0 | 5 |
| GP-1 | 1.5 | 1.5 | 1.5 | 1.5 |
| EB-21 | 1 | 1 | 1 | 1 |
| CARBINOL FLUID | 97.5 | 92.5 | 92.5 | 92.5 |
| TOTAL | 100 | 100 | 100 | 100 |
| Physical property | HARD | HARD | HARD | HARD |
| | GEL | GEL | GEL | GEL |
| STICK HARDNESS (gf) | 35 | 46 | 45 | 38 |

| INCI Name | Ex 8 | EX 9 | EX 10 |
|---|---|---|---|
| PROPYLENE GLYCOL | 10 | 15 | 20 |
| GP-1 | 1.5 | 1.5 | 1.5 |
| EB-21 | 1 | 1 | 1 |
| CARBINOL FLUID | 87.5 | 82.5 | 77.5 |
| TOTAL | 100 | 100 | 100 |
| Physical property | HARD GEL | HARD GEL | HARD GEL |
| GEL HARDNESS (gf) | 307.7 | 259.3 | 272.5 |

1. Heated glycol to around 100° C., then added gelators GP-1 and EB-21 gradually during mixing until they were dissolved to form a clear solution.
2. In the other beaker, heated the Carbinol fluid to 100° C.
3. Poured carbinol fluid into beaker containing gelator solution at temperature of 100° C. and mixed until it became homogeneous.
4. Poured the hot solution into the mold. Once the solution set in the mold, put the mold in the freezer for about 30 minutes.

### Example 11

### Clear Gel Stick Formulation

| INCI Name | Ex 11 |
|---|---|
| BUTYLENE GLYCOL | 3 |
| GP-1 | 2 |
| EB-21 | 2 |
| CARBINOL FLUID | 33 |
| Octododecanol | 30 |
| Octododecyl neopentanoate | 30 |
| TOTAL | 100 |

The resulting gel stick is clear and has a good hardness.
1. Heated glycol to around 100° C., then added gelators GP-1 and EB-21 gradually during mixing until they were dissolved to form a clear solution.
2. In the other beaker, heated the Carbinol fluid, octododecanol and octododecyl neopentanoate to 100° C.
3. Poured heated oils in step 2 into a beaker containing gelator solution at temperature of 100° C. and mixed until it became homogeneous.
4. Poured the hot solution into the mold. Once the solution set in the mold, placed the mold in the freezer for about 30 minutes.

### Colored Compositions and Processes

### Example 12

### Gel Stick

| Phase | INCI Name | Ex 12 |
|---|---|---|
| A | BUTYLENE GLYCOL | 3 |
| A | dibutyl lauroyl glutamide (GP-1/Ajinomoto) | 2 |
| A | dibutyl ethylhexanoyl glutamide. | 2 |
| | (EB-21/Ajinomoto) | |
| B | Bis-hydroxyethoxypropyl Dimethicone | 46 |
| | (CARBINOL FLUID) | |
| B | Octododecanol | 10 |
| B | cyclopentasiloxane | 35 |
| B | Nylon-611/Dimethicone | 2 |
| | TOTAL | 100 |

### Procedure:

1. Heated butylene glycol to around 100° C., then added gelators GP-1 and EB-21 gradually during mixing until they were dissolved to form a clear solution.
2. In the other beaker, phase B was prepared by heating the carbinol and octododecanol oils to 100° C. and then dissolved the Nylon-611/dimethicone copolymer.
3. Once both phases were homogenous, combined and mixed the two until they became homogeneous.
4. Poured the hot solution into the mold. Once the solution set in the mold, put the mold in the freezer for about 30 minutes. The resultant product was a Stick in hard gel form.

### Example 13

### Foundation Stick

| Phase | INCI Name | Ex 13 |
|---|---|---|
| A | BUTYLENE GLYCOL | 5 |
| A | dibutyl lauroyl glutamide (GP-1/Ajinomoto) | 3 |
| A | dibutyl ethylhexanoyl glutamide (EB- | 2 |
| | 21/Ajinomoto) | |
| B | Bis-hydroxyethoxypropyl Dimethicone | 10 |
| | (CARBINOL FLUID) | |
| B | Dimethicone (1.5 cst) | 21 |
| B | Dimethicone (5 cst) | 7 |
| B | cyclopentasiloxane | 43 |
| B | Nylon-611/Dimethicone Copolymer | 2 |
| | (Dow Corning) | |
| B | Octododecyl Neopentanoate | 10 |
| B | Diisostearyl malate | 10 |
| B | Polyglyceryl 2-diisostearate | 10 |
| C | Pigments | 18 |
| | TOTAL | 100 |

### Procedure:

1. Heated butylene glycol to around 100° C., then add gelators

GP-1 and EB-21 gradually during mixing until they were dissolved to form a clear solution.
2. In the other beaker, phase B was prepared by heating the oils to 100° C. and then dissolved the Nylon-611/dimethicone copolymer.
3. Once both phases were homogenous, combined and mixed the two until they became homogeneous.
4. Once both phases were homogenous, combined and mixed the two. Poured the beaker component B into beaker component A at temperature of 90 C and mixed until it became homogeneous.
5. Pigments in phase C was added and mixed.
6. Poured the hot solution into the mold. Once the solution set in the mold, put the mold in the freezer for about 30 minutes. The resultant product was a Stick in hard gel form.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A non-aqueous composition that is capable of forming a gel structure, comprising (a) a low molecular mass N-acyl glutamic acid diamide having a straight-chain alkyl group; (b) a low molecular mass N-acyl glutamic acid diamide having a branched-chain alkyl group; (c) at least one gel-promoting solvent which includes a substituted hydrocarbyl functional siloxane and/or a second, different gel-promoting solvent; (d) at least one active ingredient, and (e) optionally, at least one polyorganosiloxane-containing polymer; wherein the composition has a hardness value ranging from about 30 to about 300 gf, a melting point of about 50° C. or higher.

2. The composition of claim 1 wherein (a) is dibutyl lauroyl glutamide.

3. The composition of claim 1 wherein (a) is employed in an amount of from about 0.1 to about 10% by weight, based on the weight of the composition.

4. The composition of claim 1 wherein (b) is dibutyl ethylhexanoyl glut amide.

5. The composition of claim 1 wherein (b) is employed in an amount of from about 0.1 to about 10% by weight, based on the weight of the composition.

6. The composition of claim 1 wherein the substituted hydrocarbyl siloxane gel-promoting solvent is Bis-hydroxyethoxypropyl Dimethicone.

7. The composition of claim 6 wherein the siloxane is employed in an amount of from about 10 to about 98% by weight, based on the weight of the composition.

8. The composition of claim 1 further comprising the second gel-promoting solvent which is chosen from octododecanol, a C2-C5 glycol and mixtures thereof.

9. The composition of claim 8 wherein the C2-C5 glycol is employed in an amount of from about 1 to about 20% by weight, based on the weight of the composition.

10. The composition of claim 1 wherein the composition further comprises an auxiliary solvent.

11. The composition of claim 1 wherein (a) and (b) are employed in a total amount of less than 7% by weight, based on the weight of the composition, and the composition is transparent in appearance.

12. The composition of claim 1, which contains a colorant and is colored in appearance.

13. The composition of claim 1, which comprises the polyorganosiloxane-containing polymer.

14. The composition of claim 13, wherein the polyorganosiloxane-containing polymer is Nylon-611/dimethicone copolymer.

15. The composition of claim 1, which is wax-free.

16. A process for making a non-aqueous composition that is capable of forming a gel structure, comprising:
(a) providing (a) a low molecular mass N-acyl glutamic acid diamide having a straight-chain alkyl group; (b) a low molecular mass N-acyl glutamic acid diamide having a branched-chain alkyl group; (c) at least one solvent including at least one substituted hydrocarbyl functional siloxane; (d) optionally at least one additional gel-promoting solvent; (e) at least one polyorganosiloxane-containing polymer and (f) at least one active ingredient,
(b) mixing (a)-(f), at a temperature of from about 90° C. to about 125° C., to form a heated composition; and
(c) cooling the heated composition to form the non-aqueous, structured composition, wherein the composition has a hardness value ranging from about 30 to about 300 gf, and a melting point of about 50° C. or higher.
